(19) United States
(12) Patent Application Publication
(10) Pub. No.: US 2022/0370185 A1
(43) Pub. Date: Nov. 24, 2022

(54) Scaffold for Regenerative Organization of Prosthetic Organ Tissue and Method

(71) Applicant: Vactronix Scientific, LLC, San Antonio, TX (US)

(72) Inventors: Julio C. Palmaz, Napa, CA (US); Christian G. Palmaz, Fremont, CA (US)

(21) Appl. No.: 17/752,813

(22) Filed: May 24, 2022

Related U.S. Application Data
(60) Provisional application No. 63/192,531, filed on May 24, 2021.

(51) Int. Cl. A61F 2/00; C23C 14/14

(52) U.S. Cl. CPC A61F 2/0077; C23C 14/14; A61F 2002/0086; A61F 2240/001

(57) ABSTRACT

Prosthetic scaffold for regenerative organization of organ tissue for organ function supplementation or replacement and a method for making the prosthetic scaffold by physical vapor deposition of same.

### CROSS-REFERENCE TO RELATED APPLICATIONS

The present application is claims priority to co-pending U.S. Patent Application Ser. No. 63/192,531, filed May 24, 2021.

### BACKGROUND OF THE INVENTION

Except for human donor transplant, attempts at replacing organs or parts of an organ, can be classified into two main approaches, which although sharing some common aspects, are fundamentally different. One is related to Regenerative Medicine and Tissue Engineering and the other is the Artificial or Prosthetic approach. The former relies on cell lineage evolution, self-assembly and reorganization of live primordial cell lines with or without the use of differentiator promoter molecules with the goal to regenerate functional tissue structures (1,2)

The second, provides largely artificial substitutes, functionally ready to ameliorate or replace the role of an organ (3).

### SUMMARY OF THE INVENTION

The aim of this invention is to provide mechanisms for designing prosthetic organ tissue as a scaffold for regenerative organization of organ tissue for organ function supplementation or replacement.

### BRIEF DESCRIPTION OF THE FIGURES

FIG. 1 is a diagram of a radially disposed, stacked arrangement of a multi-cell prosthetic extracellular matrix (PECM) having centrally located channels.

FIG. 2 is an electron micrograph of a current commercial stent at 250× magnification illustrating high degrees of surface irregularity and multiple foreign material inclusions with elongated defects along the long axis of the struts.

FIG. 3 is an illustration of endothelial cells migrating along microscopic, high definition grooves.

FIG. 4 is an illustration depicting additive physical vapor deposited (PVD) nitinol by orderly nickel and titanium atom deposition from a high energy plasma.

FIG. 5 is an illustration depicting a conceptual prosthetic eye in which electronic output from miniature high-performance cameras elicit image perception when coupled with optic nerve tissue using the PECM of the present invention.

### DETAILED DESCRIPTION OF THE PREFERRED EMBODIMENTS

For purposes of clarity, the following terms used in this patent application will have the following meanings:

The terminology used herein is for the purpose of describing example embodiments only and is not intended to be limiting. As used herein, the singular forms “a,” “an,” and “the” may be intended to include the plural forms as well, unless the context dearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the order discussed or illustrated, unless specifically identified, as an order of performance, It is also to be understood that additional or alternative steps may be employed.

When an element or layer is referred to as being “on,” “engaged,” “connected,” or “coupled” to or with another element, it may be directly on, engaged, connected or coupled to the other element or layer, or intervening elements or layers may be present. In contrast, when an element is referred to as being “directly on,” “directly engaged to,” “directly connected to,” or “directly coupled to” or with another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below”, or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

“Substantially” is intended to mean a quantity, property, or value that is present to a great or significant extent and less than, more than or equal to total. For example, “substantially vertical” may be less than, greater than, or equal to completely vertical.

“About” is intended to mean a quantity, property, or value that is present at ±10%. Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range, including endpoints given for the ranges.

Recitation of ranges of values herein are merely intended to serve as a shorthand method of referring individually to each separate value falling within the recited range, unless otherwise indicated herein, and each separate value is incorporated into the specification as if it were individually recited herein.

References to “embodiment” or “variant”, e.g., “one embodiment,” “an embodiment,” “example embodiment,” “various embodiments,” etc., may indicate that the embodiment(s) or variant(s) of the invention so described may include a particular feature, structure, or characteristic, but not every embodiment necessarily includes the particular feature, structure, or characteristic. Further, repeated use of the phrase “in one embodiment,” or “in an exemplary embodiment,” do not necessarily refer to the same embodiment or variant, although they may.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

The term “material” is intended to refer to elemental metals, alloyed metals or pseudometals.

For purposes of this application, the terms “pseudometal” and “pseudometallic” are intended to mean materials which exhibit material characteristics substantially the same as metals, Examples of pseudometallic materials include, without limitation, composite materials, polymers, and ceramics. Composite materials are composed of a matrix material reinforced with any of a variety of fibers made from ceramics, metals, carbon, or polymers.

As used in this application the term “layer” is intended to mean a substantially uniform material limited by interfaces between it and adjacent other layers, substrate, or environment. The interface region between adjacent layers is an inhomogeneous region in which extensive thermodynamic parameters may change. Different layers are not necessarily characterized by different values of the extensive thermodynamic parameters but at the interface, there is a local change at least in some parameters. For example, the interface between two steel layers that are identical in composition and microstructure may be characterized by a high local concentration of grain boundaries due to an interruption of the film growth process. Thus, the interface between layers is not necessarily different in chemical composition if it is different in structure.

The term “build axis” or “build direction” is intended to refer to the deposition axis in the material. For example, as a material is being deposited onto a substrate, the thickness or Z-axis of the material being deposited will increase, this is the build axis of the material.

The terms “circumferential” or “circumferential axis” is intended to refer to the radial direction of a tubular, cylindrical or annular material or to the Y-axis of a polygonal material.

The terms “longitudinal,” “longitudinal axis,” or “tube axis” are intended to refer to an elongate aspect or axis of a material or to the X-axis of the material.

The term “bulk material” is intended to refer to the entirety of the material between its surfaces.

The term “film” is intended to encompass both thick and thin films and includes material layers, coatings and/or discrete materials regardless of the geometric configuration of the material.

The term “thick film” is intended to mean a film or a layer of a film having a thickness greater than 10 micrometers.

The term “thin film” is intended to mean a film or a layer of a film having a thickness less than or equal to 10 micrometers.

The terms “physical vapor deposition” and/or is acronym “PVD” is intended to encompass sputtering, electron-beam deposition, hot-boat evaporation, reactive evaporation, ion platting, plasma sputtering and/or ion beam sputtering.

It may be argued that artificial replacements have had a broader impact in medicine, in part due to their longer history of development and because they have readily benefited from the latest technological advances in materials science, fabrication and microelectronics. Implantable devices are currently common tools of practice in several medical specialties: Ocular lens implants have been time-proven in ocular surgery (4) and cochlear implants are becoming increasingly sophisticated (5). Cardiac pacemakers and ICD (implantable cardioverter defibrillator), are probably the most successful standalone microelectronic implants so far (6). In orthopedic surgery, rehabilitation of knee function due to osteochondral degenerative changes currently uses both approaches: intra capsular introduction of cartilage building promoters (7) and total artificial joint replacement (8). Given the importance of life-style changes in patients with knee joint disease and the need to quickly restore them to deambulation, the prosthetic approach has been the most widely adopted. Other prosthetic joint substitutes like the artificial hip joint, have reached state-of-the art status, with high safety and efficacy (9). Osteo-integrated dental implants have significantly advanced prosthetic dentistry (10

Implantable cardiovascular prosthetics have been very effective and enjoy a long history of successful development: Surgical and transluminal vascular grafts and stent grafts and prosthetic cardiac valves (11), caval filters (12) coronary stents (13,14), patent foramen ovale occluders (15), atrial appendage closure devices (16) mitral valve repair clip (17), transluminal aortic valve replacement (18) among many others, already available or in development.

Regenerative strategies have been attempted in the cardiovascular system, but they have not been quite as successful as prosthetic devices. Cardiac muscle regeneration was attempted with intracoronary infusion of myoblasts (19). Also, cardiac muscle injection of progenitor cells was evaluated in patients as a therapy for no-option angina (20). The improvements achieved with these approaches have been modest at best and inconclusive insofar the claimed mechanism of action (21). Myocardial revascularization with gene therapy promoting angiogenesis (22) or implantation of endothelial progenitor cells (EPG's) has yielded modest or controversial results (23).

Bioresorbable vascular stents (BRS) can be considered a regenerative approach insofar as the theory upon which they are based. Founded on the assumption that the diseased vascular wall tissues would reorganize into functional structures, vasomotion and positive remodeling was expected concurrently with the disappearance of the scaffold (24). Although the BRS proved equivalent to drug eluting metal stents in pivotal randomization trials (25) they have been clinically abandoned as post-approval clinical evidence cast doubts about their safety and efficacy (26). Some late thrombotic events with the BRS coincided in time with complete scaffold reabsorption supporting the notion that a permanent stent scaffold may be needed to replace the loss of the arterial connective structure. This is an important component of atherosclerotic vascular occlusive disease as the vessel structure supports endothelial cell function and stability (27).

### Progenitor Stem Cells as the Building Blocks for Regenerative Therapies of Solid Organs.

Progenitor stem cells (PSC) from fetal tissues, umbilical cord, bone marrow, or fibroblast cell lines (28) can be induced to produce mature lineage cells as neural, hepatic, cardiac, renal and so forth (29). This brought about a large and ongoing body of research based on the possibility of regeneration of organs for in vitro or in vivo applications (30).

At present, the creation of fully functional isolated organs from progenitor cells appears rather remote from current perspective (31,32), but substantial work is being done with infusion of progenitor cells in diseased organs aiming at replacing cellular loss. While there are currently more than twelve hundred mesenchymal stem cell (MSC) clinical trials (Clinical trials.gov) large scale, controlled clinical studies have often failed to substantiate the benefit suggested by smaller trials (33).

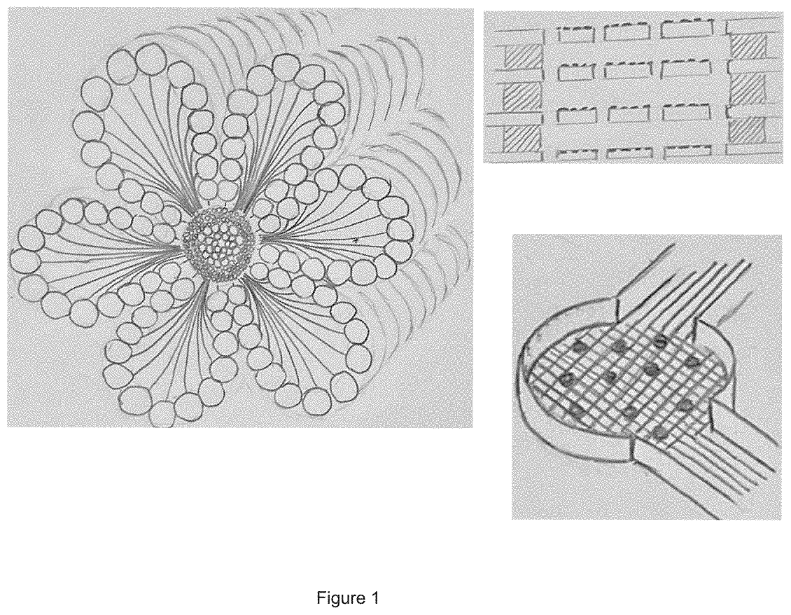

The co-location of constituent cells in the correct position and arrangement in an artificial or biological matrix has been attempted by recellularization of decellularized organs like lungs (34) and cardiac valves (35). Work with 3D printing of cells and viscoelastic matrices is progressing through preliminary stages (36). The use of porous hydrogels or bioresorbable materials such as polyglycolic acid as tissue scaffolds rely on the expectation that progenitor cells will home, assemble, differentiate and promote the formation of their own support infrastructure including vascular supply and lymphatic/venous drainage among other tissue architectural features. Based on currently available experimental evidence, it seems that the expectation of spontaneous generation of tissue infrastructure may be over optimistic. Engineered prosthetic cellular scaffolds may be more realistic tissue platforms as conceptualized by constructs such as suggested in FIG. 1. FIG. 1 illustrates a radially disposed, stacked arrangement of a multicellular prosthetic extra cellular matrix (PECM) in accordance with the present invention. The multi-cellular PECM has centrally located channels that extend along a longitudinal axis of the PECM for vascular supply to and drainage from the cells. Cellular guidance and location may be facilitated by microgrooved channels. Fluids, solutes, and molecular exchange is promoted by Z-axis channels.

It will be understood that alternative architectures of PECM arrangements are both intended and contemplated by the present invention other than the specific example illustrated in FIG. 1, including, for example compartmentalized multi-cellular structures in which the compartments are capable of communicating fluids, solutes, and molecular exchange between compartments.

Prosthetic scaffolds may be more plausible as engineered extracellular matrices may guide cell growth, location and differentiation to achieve the goal of functional semisynthetic organs. As these fledging efforts progress through basic science proof of concept to eventual practical reality, it is not inconceivable that future replacement organs will be bioprosthetic constructs rather than all-biological laboratory organs ready to be surgically implanted like donor allografts. Practically speaking, prosthetic constructs have logistical advantages compared to biological constructs insofar control of measurable parameters, fabrication techniques and reproducibility. Controlled progression to increasing complexity is another advantage of prosthetics and may translate into more predictable and consistent results.

### Organ Transplantation:

Organ transplantation of live or decellularized tissue may not be considered regenerative therapy since the transplanted organ or parts of an organ, whether it be a homograft, allograft or xenograft is functionally developed and aims at readily substitute a diminished or absent function. Optimally, live donor transplant is well tolerated by the recipient provided MEW, HLA, ABO compatibility, adjuvant chemotherapy and prevention of infection and other potentially adverse events (37). Although not a solid organ, allogenic or autologous transplants of blood precursors in the bone marrow routinely restore blood cell production in patients with bone marrow depletion by chemo or radiation therapy. However, blood producing bone marrow cells are among human tissues of rapid growth that provide for the attrition of their end-of-the-line constituents and, their spontaneous differentiation is actually pre-determined, not manipulated and, or induced. Same applies to skin and biological cardiac valves. Corneal transplants, ligaments, bone and cartilage grafts should be considered developed tissues with full functional capability like the lung, pancreas, kidney, liver and heart. Therefore, they fall outside of the frame that define regenerative therapies.

### From Scaffolds to Prosthetic Extracellular Matrix (PECM)

To create tissue scaffolds whose function would approximate the biological extracellular matrix (ECM) paradigm changes in understanding, design and fabrication methods must be made. In regard to structures build with prosthetic biomaterials, most work has been within the macroscopic or microscopic realm. Technologies such as fabrication methods, biomechanical performance and quality assurance apply largely to the visible size range with or without the assistance of optical magnification. The optimal range of most technologies used in the medical device industry is at the sub-millimeter and micron level as defined by industrial manufacturing tolerances. This may be adequate for current device technology but insufficient for sub-micron and nanometer detail.

While pharmacological agents are designed focusing on molecular interactions with cell membrane receptors and other nanoscopic size targets, implantable biomaterials interaction with biological tissues are usually assessed by histopathological effects such as mechanical injury and surface boundary phenomena such as thrombogenicity and inflammation. The focus should shift from the micron to the nanometer world for the assessment of material interactions with living tissues.

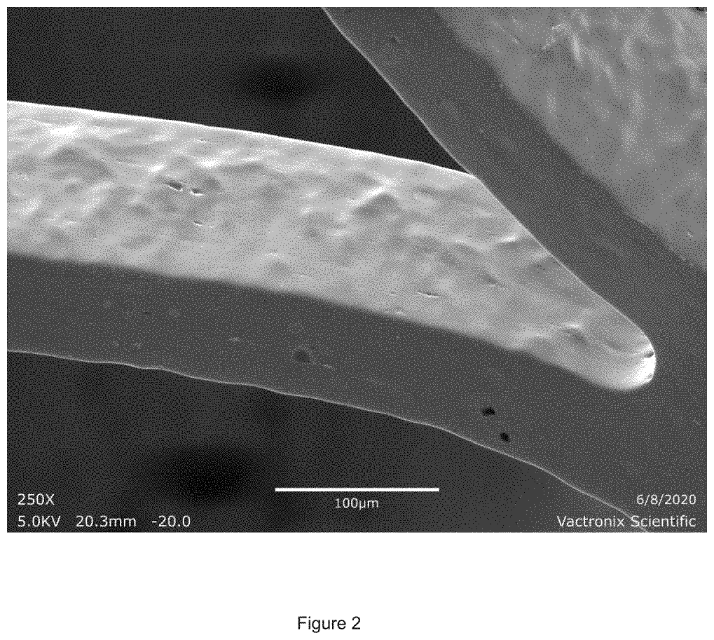

It is known, for example, that current commercial biomaterials, such as those used to fabricated vascular stents, have high degrees of surface irregularities at the microscopic level and multiple foreign material inclusions, such as carbide inclusions or residual from process lubricants. FIG. 2 is an electron micrograph illustrating such features in a commercially available vascular stent. It has been demonstrated that such surface irregularities and foreign material inclusions are impede or even prevent cellular attachment and proliferation on the material surfaces resulting in either incomplete or non-incorporation of the devices into the biological tissue. As such, current materials are sub-optimal as prosthetic tissue scaffolds, particular for organ tissue regeneration.

As progress is made into a smaller dimensional world, each step down creates new challenges. This is already evident in the quality assurance and lower tolerance levels of implantable microdevices, requiring increasingly sophisticated measuring equipment and computer data management to cope with the ever increasing design complexity and volume of QA data.

The commitment to design and manufacture at the nano-scale is neither simple nor economically efficient in the present reality of the medical device industry. However, efforts must be undertaken to change current simplistic approaches to new devices. This is not easy because approved devices are currently considered safe and effective by older stablished criteria. However, adherence to these criteria is a hinderance to progression to more sophisticated devices and even unforeseen new applications.

A PEEK into Cellular Response to Engineered Surfaces

Controlled nanofabrication on artificial surfaces allows for micro-engineering of prosthetic surfaces in a manner that is highly controllable. Working on micro-engineering of the surface of implantable vascular stents materials new techniques for fabrication of high definition microgrooves have been developed (38). The goal was to increase the migration speed and influence growth direction of human aortic endothelial cells to accelerate colonization and therefore positively influence the prosthetic healing process. Engineered micro-grooves have been made that are within the size range of vascular endothelial cells (EC). An effort was made to maintain the features definition within few hundred nanometers. As technology moved from abrasive to laser, to photolithography manufacturing techniques it was quickly realized that the limiting factor was the materials being working with. The engineered features on the prosthetic surface that were initially used were within the size range of currently accepted surface topography variability. This implied that to fabricate those features with consistent regularity the putative background surface had to be chemically and topographically more uniform. The result of trying many iterations of fabrication techniques and material, revealed the importance of the edge definition of the features. In other words, the cellular response increased with control of feature regularity at submicroscopic level. Migrating endothelial cells are sensitive to surface topography whether this is by their physical presence or by redistribution of surface adhesive proteins. Un-interrupted outer edges and inner corners act as guides for the EC lamellipodia and other migrating cell filopodia to follow, pulling the rest of the cell behind.

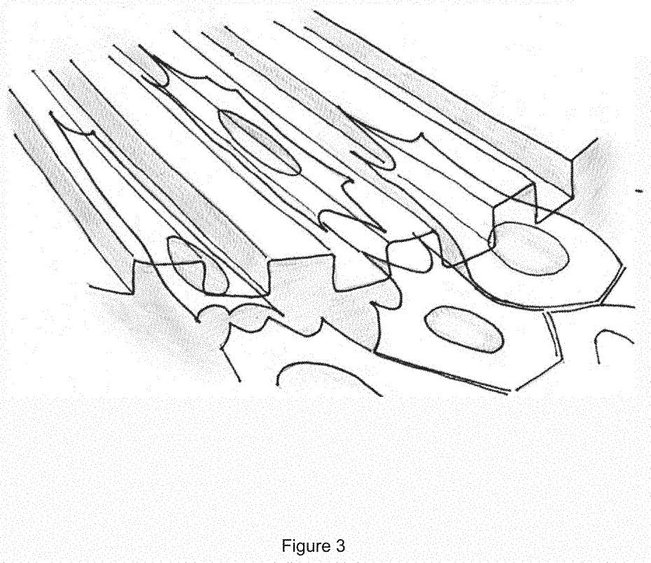

FIG. 3 depicts endothelial cells migrating along microscopic, high definition grooves in surfaces of biomaterials such as those used in the disclosed organ tissue scaffolds of the present invention. The grooves direction guide the migration of the cells, increasing their translation speed and their ability to more rapidly colonize a surface.

This guidance effect is lost as the feature edge gets interrupted. Thereby, the importance of high-definition longitudinal feature continuity. Guidance effects can be an approach to correctly locate cells within a matrix whether they be endothelial, epithelial, parenchymal or osteoblastic cellular arrays, or attempts at neuroaxonal reconnection. This pursuit necessitates the development of new biomaterials to allow fabrication in the sub-microscopic range. By achieving nanoscopic controlled features a surprising and unexpected change in cellular response from random to consistent was observed. Higher quality levels of material composition defined as topographical and chemical homogeneity and enhanced robustness provide an adequate canvass for new, high-definition designs and fabrication. Currently, the metal alloys used for most implantable devices have marked crystal size variability and carry lattice defects and impurities in the form of inclusions from fabrication residues (39). These materials cannot be used to make device features with sub-microscopic detail. This is also important with device features smaller than the size of a single crystal or inclusion deposit in current bio-alloys as corrosion and fatigue resistance quickly diminish with miniaturization.

As prosthetic scaffolds evolve toward microscopic designs, the architecture of the PECM to favor cell attachment mechanisms, fluids exchange, large and small molecular movement, immune cell migration and apoptotic cell debris disposal (FIG. 1, PECM construct) must be addressed and considered. With the development of microscopic intricate designs, the material-cell surface interface will increase exponentially to the point that physical chemistry properties of the material will become critical. Probably, current levels of acceptance to ion release, surface hydropathy, electrical charge, stability of surface oxides and other surface properties will become central as they determine selective adhesive protein distribution and interaction with surfaces. Like with current designs, resistance to corrosion, elasticity, strength and fatigue limits, will be dependent on each application. But, given the intricacy and complexity of future designs, assessment of biomechanical parameters will become more difficult. Nonetheless, these complex constructs may be surprisingly robust as prosthetic/living tissue association may introduce new aspects of composite materials engineering, previously ignored or underestimated.

### A New Manufacturing Environment

Current devices are largely made by top-down or reductive manufacturing. These classical technologies rely on extensive and skilled hand labor, produce waste and suffer from relatively large product variability and rejection rates. Reductive manufacturing commonly involves a complicated series of steps, each one leaving footprints that affect the surface chemical and topographical homogeneity. As complex microscopic and submicroscopic constructs are considered, these methods may no longer be useful. Human manual processing will be replaced by automated manufacturing and new technologies uncommon in the medical device industry today will be common place. This is the case for physical vapor deposition (PVD) of metals and alloys, ceramics and polymers and extensive robotic use of photolithography and femto-second lasers.

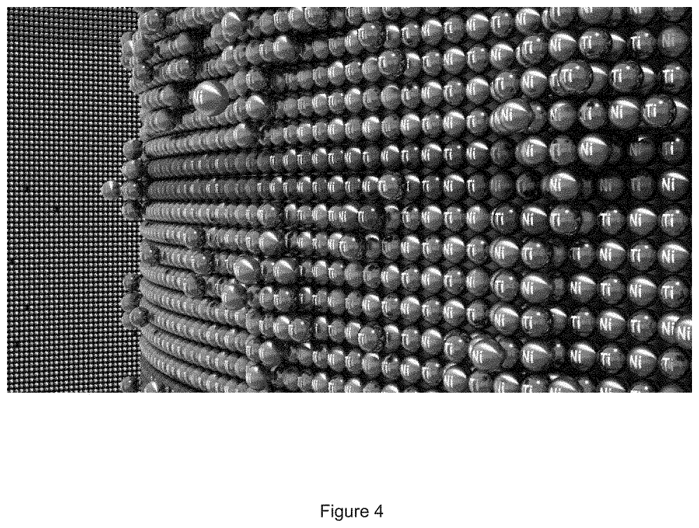

Additive PVD achieves an orderly deposition of metal or pseudometal atoms from a high energy plasma. FIG. 4 is an illustration at of this orderly deposition of nitinol from nickel and titanium atoms. PVD process are highly controllable to yield predictive material properties in the resulting deposited material. Moreover, the structures of the deposited materials may have complex geometries based upon the geometry of the substrate onto which the deposition occurs. Thus, the cellular scaffold elements of the inventive PCM may be formed by PVD processes by depositing the desired metal or pseudometal onto substrates having geometries corresponding to the desired shape of the cellular scaffold elements.

The enabling technologies are today more ubiquitous in energy and electronics fields than in medical devices. Most importantly, quality assurance and control requirements will exponentially increase with complex, smaller devices, requiring larger computer data processing. The use of machine learning and artificial intelligence (AI) will allow for an increase in the role of high-power computers in all aspects of new devices from complex designs, to manufacturing, to quality control and data handling. The new manufacturing facilities will likely have smaller physical plants, fewer operators and substantial computer involvement.

The evolution of technology toward fabricating features dimensionally closer to the size of cells is inevitable but the technology will change to the point it does no longer resemble what is a typical medical device company. The regulatory agencies may need to adapt to the change by shifting the emphasis from device-centered to method-centered, thereby regulating the certification of processes that allow for flexibly changing design and eventually facilitating personalized device manufacturing.

In some ways, future creation of new devices will resemble new drug design and manufacturing that focus on subcellular targets. Surface chemistry at device surfaces as well as surface topography at the molecular level and its effect on and interaction with cells and molecules must be considered. This calls for a fundamental change in the way prosthetic constructs and design will address the molecular make-up of the biological environment rather than using the current adaptive/adoptive approach. In other words, no longer available materials and technologies will be borrowed from other fields, based on their procure-ability, practicality and implementation by trial and error. Rather, materials and methods will be tailor-made to fit applications.

Eventually, fabrication of devices will be chemical-based. Partly in-vitro and partly in-vivo, the assembly of microscopic components will be managed by complementary reactants strategically placed in nanoblocks with geometrical growth capability. Architectural plasticity will be managed by computer control of reactants acting as chemical triggers of hierarchical in-vivo growth. Carbon nanoparticles maybe possible candidates for their material properties and the ability for chemical derivatization. However fascinating, chemical based in-vivo prosthetic build-up is farther in the future that the possibilities presently at hand. (40).

What will be the next big step in the discovery and application of organ replacements is hard to predict as so many factors influence these evolutionary trends. However, some pieces of this puzzle are already in place providing a glimpse of what may be the next big thing in this area. Starting with prosthetic biomaterials, the use of AI to create super-materials may eliminate what is today a long and tedious process to arrive at products with ideal properties by trial and error. Computers will tell us the ideal combination of components with perfect stoichiometry for optimized properties for a given application (41). Superalloys will be made overnight in vacuum plasma reactors rather than in foundry furnaces. Manufacturing labor, waste and rejection of out of specification product will be almost eliminated.

Progress in understanding tissue transmission of electrical and electromagnetic energy (42) is already influencing implantable technologies like cardiac pacing, in the application of addressable lead-less pacemakers (43). Also, it should aid in new developments in neuroscience technology to successfully establish connectivity between electrical impulses originated in the brain and external actuators, or from impulses generated extracorporeally to elicit specific neuronal excitation (44). This will open up long-awaited opportunities for rehabilitating victims of traumatic and degenerative brain-spinal disfunction resulting in enabled motor function and speech. It is not hard to imagine that if brain connectivity is finally conquered, vision and auditory functions can also be restored. Furthermore, with current available technologies is easy to imagine enhanced sensory functions as high definition, infrared and ultra-low light vision.

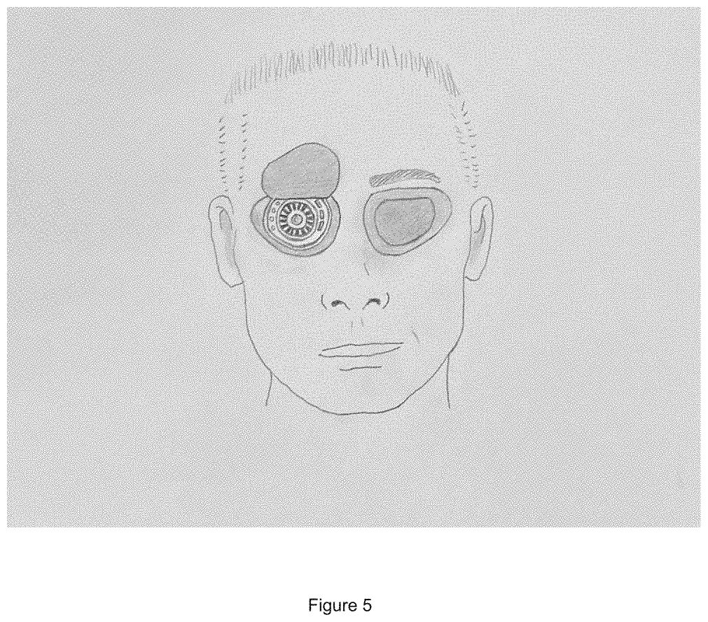

FIG. 5 illustrated a conceptual depiction of a prosthetic eye that employs high-performance cameras as part of the eye prosthesis. The electrical output from the cameras must communicate with the optical neural pathways. Such communication is made possible by configuring a multi-cellular PECM of the present invention as a optical neural pathway from the prosthetic eye to the optic nerve or other neural pathway to the brain.

This same principal applies to all sensory organs, such as enhancing or replacing pathways for touch, taste, smell, hearing or the like. Following this line of thought is also conceivable that radio communications allowed by an effective computer brain-interface will provide access of just about all the telecommunications and internet connectivity that most human beings presently enjoy by mere conscious thinking.

### The Social Impact of Prosthetic Replacements

The improvement in quality of life that many prosthetic devices offer to the aging population are amazing. This is mainly by alleviating life-style limiting conditions that were previously ostracizing and, or debilitating and contributed to the inevitable decline of old age.

However, more significant is the progressive disappearance of the prosthetic stigma. The ancient prototypical wooden leg, ear horn and the gold tooth have given place to high tech limb prosthesis and imperceptible tooth implants. Just as important as the functional benefit they provide, is the decrease of real or perceived social isolation imposed by the need of using odd prosthetic replacements or aids. It can easily be imagine that if enhanced vision and other sensory functions become available, the level of acceptance will be even higher or even be replaced by a sense of pride in the user. Electro-mechanically assisted exo-skeletal frames are commonplace in assembly lines to assist high effort operations (45). It is not inconceivable that they will replace the traditional wheel-chair, particularly if they can be operated by violative brain command.

In other words, the lines between handicapped and non-handicapped people may start blurring out and many of these life-facilitating and life-enhancing prosthetic assists may have profound influences in society.

### REFERENCES

1. Meritxell Huch, Juergen A. Knoblich, Matthias P. Lutolf, Alfonso Martinez-Arias The hope and the hype of organoid research Development 2017 144: 938-941
2. G Rossi, A Manfrin, M Lutolf. Progress and potential in organoid research. Nature Reviews Genetics 19, 671-687; 2018
3. B Ratner, A Hoffman, F Schoen, J Lemons. Biomaterials Science 2^{nd} ed. Elsevier

Academic Press, 2004
4. D Allen, A Vasavada. Cataract and surgery for cataract. BMJ 2006, 333:128-132
5. Lenarz. Chloclear implant-state of the art. GMS Curr Top Otorhinolarygol Head Neck Surg 2017; 16:Doc
6. Trohman R, Kim M, Pinski S. Cardiac pacing: State of the art. The Lancet 2004; 364(9446):1701-19
7. K Filardo, G Roffi, A Andriolo, L Marcacci M. New trends for knee cartilage regeneration: From cell-free scaffolds to mesenchymal stem cells. Curr Rev Musculoskelet Med 2012; 5:236
8. J Feng, D Novikov, A Anoushiravani, Ran Schwarzkopf. Total knee arthroplasty:

Improving outcomes with a multidisciplinary approach. J Multidiscip Health 2018; 11:63
9. U Holzwarth, G Cotogno. https://ec.europa.eu/jrc/en/publication/eur-scientific-and-technical-research-reports/total-hip-arthroplasty-state-art-prospects-and-challenges).
10. D Hong, J Oh. Recent advances in dental implants. Maxillofac Plast Reconstr Surg 2017; 39(1):33
11. F Johnson, K Virgo. The bionic human. Humana press inc. 2006. NJ 07512. Chapter 18, p 641
12. E De Young, J Minocha. Inferior vena cava filters: Guidelines, best practice and expanding indications. Sem Intervent Radiol 2016; 33 (2):65-70
13. R Schatz, Goldberg S, M Leon, et al. Clinical experience with the Palmaz-Schatz stent. JACC 1991 1991; 17(6):155B-9B. D Fischman, D Baim, R Schatz, et al. A randomized comparison of coronary-stent placement and balloon angioplasty in the treatment of coronary artery disease. NEJM1994; 331:496-501.
14. P Serruys, P de Jaegere, F Kiemeeneij, et al. A comparison of balloon-expandable-stent implantation with balloon angioplasty in patients with coronary artery disease. NEJM1994; 331:489-495
15. F Collado, M Poulin, J Murphy et al. Patent foramen ovale closure for stroke prevention and other disorders. JAHA 2018; 7e007 146. DOI:10.1161/JAHA.117.007146
16. G Casu, G Gulizia, G Molon, et al. ANMCO/AIAC/SICI-GISE/SIC/SICCH consensus document: percutaneous occlusion of the left atrial appendage in non-valvular atrial fibrillation patients:indications, patient selection, staff skills, organization and training. European Heart Journal Suppl 2017. 19 (suppl D), D333-D353
17. N Oh, P Kampaktsis, M Gallo, et al. An updated meta-analysis of MitraClip versus surgery for mitral regurgitation. Ann Cardiothorac Surg 2021; 10(1):1-14
18. V Kumar, G Sanhdu, C Harper et al. Transcatheter aortic valve replacement programs: Clinical outcomes and developments. J Am Heart Assoc.2020:e015921.DOI:10.1161/JAHA.120.015921
19. K Suzuki, B Murtuza N Suzuki, R Smolenski, M Yacoub. Intracoronary infusion of skeletal myoblasts improves cardiac function in Doxorubicin-induced heart failure Circulation 2001; 104(I):I-213-217
20. T Henry, Losordo D, J Traverse, R Schatz, et al. Autologous CD34 cell therapy improves exercise capacity, angina frequency and reduces mortality in no-option refractory angina: a patient-level pooled analysis of randomized double-blinded trials. European Heart J 2018; 39:2208-2216
21. L He, N Nguyen, R Ardehali, B Zhou. Heart Regeneration by endogenous stem cells and cardiomyocyte proliferation. Controversy, fallacy and progress. Circulation 2020; 142:275-291). (M Seidel M, A Borczynska, N Rozwadowska, M Kurpisz. Cell-based therapy for heart failure: Skeletal myoblasts. Cell Transplantation 2009; 18:695-707
22. S Yla-Herttuala, C Bridges, M Katz, P Korpisalo. Angiogenic gene therapy in cardiovascular diseases: dream or vision? European Heart Journal 2017; 38:1365-1371
23. S Aurangzeb, E Shantzila, G Lip, C Varma. Endothelial progenitor cells: What use for the cardiologist? J Angiogenesis Res 2010; 2(1):6). (J Pearson. Endothelial Progenitor cells-hype or hope? J Thromb Haemost, 2009; 7:255-62
24. J Lane, L Perkins, A Sheehy, et al. Lumen gain and restoration of pulsatility after implantation of a bioresorbable vascular scaffold in porcine coronary arteries. JACC: Cardiovascular Interventions 2014; 7(6):688-695
25. G Stone, R Gao, T Kimura, et al. 1 year outcomes with the Absorb bioresorbable scaffold in patients with coronary artery disease: a patient-level, pooled meta-analysis. Lancet 2016; 387:1277-89
26. D Kereiakes, S Ellis, C Metzger, et al. 3-year outcomes with everolimus-eluting bioresorbable coronary scaffolds. The absorb III trial. JACC 2017; 70(23):2852-62
27. S Dhawan, R Nanjundappa, J Branch, R Taylor, et al. Shear stress and plaque development. Expert Rev Cardiovasc Ther. 2010; 8(4): 545-556
28. T Ichim, P O'Heeron, S Kesan. Fibroblasts as a practical alternative to mesenchymal stem cells. J Transl Med 2018; 16:212
29. E Yang, N Liu, Y Tang et al. Generation of Neurospheres from human adipose-derived stem cells. Biomed Res. Int. 2015; 2015:743714
30. T Chen, F Wang, M Wu, Z Wang. Development of hematopoietic stem and progenitor cells from human pluripotent stem cells. J Cell Biochem 2015; 116(7):1179-89
31. Meritxell Huch, Juergen A. Knoblich, Matthias P. Lutolf, Alfonso Martinez-Arias. The hope and the hype of organoid research Development 2017 144: 938-941
32. G Rossi, A Manfrin, M Lutolf. Progress and potential in organoid research. Nature Reviews Genetics 19, 671-687; 2018
33. S Ezquerra, A Zuleta, R Arancibia, et al. Functional properties of human-derived mesenchymal stem cells spheroids: A meta-analysis and systemic review. Stem Cells Int 2021:8825332), (J Galipeau, L Sensebe. Mesenchymal stromal cells:clinical challenges and therapeutic opportunities. Cell Stem Cell 2018; 1:22(6):824-833
34. T Tsuchiya, A A Sivarapatna, K Rocco, et al. Future prospects for tissue engineered lung transplantation. Organogenesis 2014; 10(2):196-207
35. A Bonetti, M Marchini, F Ortolani. Ectopic mineralization in heart valves: New insights from in-vivo procalcific models and promising perspectives on non-calcifiable bioengineered valves. J Thorac Dis 2019; 11(5):2126-2143
36. M Skylar-Scott, S Uzel, L Nam, et al. Biomanufacturing of organ specific tissues with high cellular density and embedded vascular channels. Sci Adv. 2019; 5:eaaw2459
37. M Ayala Garcia, B Yebra, A Lopez florez, E Guerra. The Major Histocompatibility Complex in transplantation. Journal of Transplantation 2012. Article ID 842141
38. E Sprague, F Tio, H Ahmed, et al. Impact of parallel micro-engineered stent grooves on endothelial cell migration, proliferation and function. Circulation Cardiovascular Interventions 2012; 5(4):499-507
39. P Adler R Frei, M Kimiecik. Effects of tube processing on the fatigue life of nitinol. Shap. Mem. Supeerelasticity (2018) 4:197-217
40. J Palmaz https://www.youtube.com/watch?v=W7JxWUoKL8
41. G Ceder, K Persson. 2013 https://www.scientificamerican.com/article/how-supercomputers-will-yield-a-golden-age-of-materials-science/42.
42. J A Tuszynski. 2018 https://www.ncbi.nlm.nih.gov/books/NBK549549/43.
43. F Tjong, V Reddy. Permanent leadless cardiac pacemaker therapy. Circulation 2017; 135(15):1458-7
44. S Begley. 2018 https://www.scientificamerican.com/article/with-brain-implants-scientists-aim-to-translate-thoughts-into-speech/45.
45. S Fox, O Aranko, J Heilala, P Vahala. Exoskeletons: Comprehensive, comparative and critical analysis of their potential to improve manufacturing performance. J Manufacturing Technology Management 2019; 31 (6):1261-8

What is claimed is:

1. A regenerative organ tissue scaffold, comprising a plurality of compartments organized in a three-dimensional array, each of the plurality of compartments being in physiological and/or biological communication with each other along at least two axes of the three-dimensional array; and a plurality of channels communicating along a Z-axis of the three-dimensional array configured for vascular supply and/or drainage from each of the plurality of compartments in the three-dimensional array.

2. A method of making a regenerative organ tissue scaffold, comprising the steps of physical vapor depositing at least two of a plurality of compartments; assembling the at least two of a plurality of compartments into a three-dimensional array; and forming a plurality of channels communicating a Z-axis of the three-dimensional array configured for vascular supply and/or drainage from each of the plurality of compartments in the three-dimensional array.
